(19) United States
(12) Patent Application Publication
(10) Pub. No.: US 2022/0313823 A1
(43) Pub. Date: Oct. 6, 2022

(54) PHOTODYNAMIC THERAPY METHOD MEDIATED BY CHLORIN E6 PHOTOSENSITIZER COMPOSITE FOR TREATING AND PREVENTING OBESITY

(71) Applicant: DONG SUNG PHARM. CO., LTD., Seongnam-si, Gyeonggi-do (KR)

(72) Inventors: Yong Wan KIM, Seoul (KR); Mi Young LEE, Seoul (KR); A Reum RYU, Asan-si (KR); Cheong Wun KIM, Daegu (KR)

(73) Assignee: DONG SUNG PHARM. CO., LTD., Seongnam-si, Gyeonggi-do (KR)

(21) Appl. No.: 17/634,487

(22) PCT Filed: May 15, 2020
(86) PCT No.: PCT/KR2020/006469

§ 371 Date: Feb. 10, 2022

(30) Foreign Application Priority Data
May 15, 2019 (KR) 10-2019-0056967
May 15, 2020 (KR) 10-2020-0058711

(51) Int. Cl. A61K 41/00; A61P 3/04

(52) U.S. Cl. CPC A61K 41/0071; A61P 3/04; A61K 9/0019

(57) ABSTRACT

Disclosed, according to one embodiment, is a photodynamic therapy method mediated by a chlorin e6 photosensitizer composite for treating and preventing obesity, the method comprising the steps of: injecting or administering a chlorin e6 composite into a subject; and performing photodynamic therapy on the subject into which the chlorin e6 composite has been injected or administered.

### TECHNICAL FIELD

The present invention relates to a photodynamic therapy method mediated by a chlorin e6 photosensitizer composite for treating and preventing obesity.

### BACKGROUND ART

Obesity is one of the causes of serious health problems worldwide due to an imbalance in energy consumption and storage above all things (Reference 1). Obesity is associated with many high-risk diseases (cardiovascular disease, diabetes, hyperlipidemia, cancer, etc.). Adipose tissue is composed of numerous adipocytes, which are known to be very important for energy metabolism homeostasis. Adipose tissue is divided into brown adipose tissue (BAT) and white adipose tissue (WAT). The WAT is used to store energy and the BAT is used to generate heat by burning fat (2).

Proliferation of adipocytes results from the production of fat and lipids, which proliferate as precursors of adipocytes are differentiated into adipocytes (Reference 3). Adipocyte differentiation proceeds through a dynamic and complex process, inducing a morphological change of cells and insulin sensitivity, and causing a continuous expression of genes transcribed and translated by secreted molecules. Peroxisome proliferator activated receptor-γ (PPARγ) and CCAAT/enhancer-binding protein alpha (C/EBPa) are important factors in regulating adipogenesis during the early stages of adipocyte differentiation (4). PPAR-γ is a main regulator of adipocyte differentiation, which is one of the most specific markers for adipogenic differentiation (Reference 5). C/EBPa is one of the proteins that exert the most harmonious effect together with PPAR-γ during adipocyte maturation and differentiation (Reference 6).

3T3-L1 adipocyte precursors are differentiated into adipocytes as glucose consumption and triglycerides increase. PPAR-γ and C/EBPa, which are expressed at the initial stage of differentiation, are also expressed as sterol regulatory element binding protein-1 (SREBP-1). This protein is one of the most important proteins in the regulation of adipogenic transcription factors and is involved in the biosynthesis of cholesterol, fatty acids, and triglycerides. In addition, lipoprotein lipase (LPL) is abundantly contained in adipose tissue and acts to catalyze the hydrolysis of triglycerides (Reference 7). The mRNA expression of this LPL is also considered as a marker to confirm the early stage of differentiation of adipocytes, and all transcription factors induce the expression of lipid synthesis genes such as fatty acid synthase (FAS) and adiponectin together (Reference 8, Reference 9). Thus, if these proteins are controlled in the early stage of lipogenesis, obesity can be prevented and related diseases can also be prevented. Accordingly, the present inventors have decided to use a photodynamic therapy (PDT) method to induce anti-obesity effect.

PDT is one of the very attractive methods for the treatment of cancer and other diseases (antioxidant, anti-inflammatory, antibacterial, anti-fungal, anti-aging, etc.), and follows a method of treating diseases by using high ROS generated through a photosensitizer that responds to a specific wavelength of laser (660 nm) (Reference 10). Out of the photosensitizers, a chlorin e6 composite is a second-generation photosensitizer with a maximum absorption wavelength of 662 nm, a penetration depth of 12 nm into treatable diseased cells, and a short time for laser irradiation after administration of the photosensitizer. Thus, the chlorin e6 composite has an advantage of shortening a period of time required for blocking light as a drug is substantially removed in up to three days.

The present invention has confirmed the expression of proteins such as PPAR-γ, C/EBPa, AMPK, LPL, and FAS in a stage of cell experiments through the PDT method using the chlorin e6 composite, and how much the white fat is reduced in a stage of animal experiments, thereby proving that the PDT method using the chlorin e6 composite has a potential anti-obesity effect.

### DISCLOSURE

### Technical Problem

According to one embodiment, the present invention may provide a photodynamic therapy method mediated by a chlorin e6 photosensitizer composite for treating and preventing obesity.

According to another embodiment, the present invention may provide a photodynamic therapy method mediated by a chlorin e6 photosensitizer composite for treating and preventing a subject's obesity and metabolic diseases.

### Technical Solution

According to one embodiment, there may be provided a photodynamic therapy method mediated by a chlorin e6 photosensitizer composite for treating and preventing obesity, the method comprising: injecting or administering a chlorin e6 composite into a subject; and performing photodynamic therapy on the subject into which the chlorin e6 composite has been injected or administered.

According to another embodiment of the present invention,

there may be provided a photodynamic therapy method mediated by a chlorin e6 photosensitizer composite for treating and preventing a subject's metabolic diseases, the method comprising: injecting or administering a chlorin e6 composite to the subject; and

performing photodynamic therapy on the subject into which the chlorin e6 composite has been injected or administered.

In the embodiments described above, the injecting or administering of the chlorin e6 composite to the subject; and the performing of photodynamic therapy on the subject into which the chlorin e6 composite has been injected or administered may be periodically carried out twice or more.

The injecting or administering of the chlorin e6 composite to the subject may be administering the chlorin e6 composite in an amount of 1.0 mg to 10.0 mg per 1 kg of the subject.

The performing of photodynamic therapy may be to expose the subject to an LED laser having a wavelength of 660 nm and an intensity of 1.0 J/cm² to 5.0 J/cm².

The chlorin e6 composite may include at least one of chlorin K, chlorin P6, rhodochlorin, perpurin 18, chlorin e4, rhodin G7, porphyrin K, and porphyrin e4 among chlorin e6 and compounds derived from chlorin e6.

The chlorin e6 composite may include at least one of chlorin K, chlorin P6, rhodochlorin, perpurin 18, chlorin e4, rhodin G7, porphyrin K, and porphyrin e4 among chlorin e6 and compounds derived from chlorin e6, and additives.

### Advantageous Effects

According to at least one embodiment of the present invention, a photodynamic therapy method mediated by a chlorin e6 photosensitizer composite for treating and preventing obesity can be effective in obesity treatment or prevention when applied to a subject.

According to at least one embodiment of the present invention, a photodynamic therapy method mediated by a chlorin e6 photosensitizer composite can be effective in treating and preventing a subject's obesity and metabolic diseases.

### DESCRIPTION OF DRAWINGS

FIGS. 1A to 1I show the structures of chlorin K, chlorin P6, rhodochlorin, perpurin 18, chlorin e4, rhodin G7, porphyrin K, and porphyrin e4 among chlorin e6 and the compounds derived from chlorin e6 which are used in a photodynamic therapy method according to one embodiment of the present invention.

FIG. 2 is a graph of confirming a viability by applying PDT using a chlorin e6 composite to 3T3-L1.

FIG. 3 is a view of showing the differentiation of 3T3-L1 adipocytes and an experimental plan for applying PDT mediated by a chlorin e6 composite.

FIG. 4 is a view of confirming the effect of PDT applied through a chlorin e6 composite on inhibiting the lipid accumulation and differentiation of 3T3-L1 adipocytes by using oil-red-O staining.

FIG. 5 is a view of confirming the effect of inhibiting the synthesis and accumulation of triglycerides in 3T3-L1 adipocytes by using nile red staining.

FIGS. 6A and 6B are views of confirming the effect of PDT mediated by a chlorin e6 composite on regulating the expression levels of adipogenic transcription factors and AMPK in differentiated 3T3-L1 cells through a Western blot method.

FIG. 7 is a view of confirming the effect of PDT mediated by a chlorin e6 composite on regulating the expression level of lipogenesis mRNA in differentiated 3T3-L1 cells.

FIG. 8 is a view of summarizing a mechanism by which PDT mediated by a chlorin e6 composite inhibits an increase in fat weight and the production of differentiated 3T3-L1.

FIG. 9 is a view of summarizing the effect of PDT mediated by a chlorin e6 composite on the WAT and BAT in mice with obesity induced.

FIGS. 10 and 11 are CT scan views for confirming whether PDT mediated by a chlorin e6 composite has an effect of ameliorating obesity in beagle dogs with high-fat diet induced obesity.

### MODE FOR INVENTION

Hereinafter, the configuration and effects of the present invention will be described in more detail through the following exemplary embodiments, but these exemplary examples are provided only for the purpose of illustrating the present invention, and thus the scope of the present invention is not limited thereto.

The terms used herein are used only for describing exemplary embodiments and are not intended to limit the present invention. In the present specification, the singular forms also include the plural forms unless specifically stated otherwise in the phrase. Terms “comprises” and/or “comprising” as used in the present specification shall not be construed to exclude a possible presence or addition of one or more other components, in addition to a mentioned component.

In the present specification, the term “treatment” is meant to include removing an advanced disease symptom or the cause of the disease symptom, alleviating the disease symptom, or suppressing the further progression of the disease symptom.

According to the present invention, chlorin e6 may be an effective photosensitizer that has been used for a long time in various skin and cancer disease models.

Chlorin may be a large heterocyclic aromatic compound centered on three pyrrole rings and one reduced pyrrole ring linked by four methine bonds. Magnesium-containing chlorin may be called chlorophyll and may be a main photosensitive pigment in the chloroplasts of most plants, algae and cyanobacteria. Chlorin was introduced as a potential photosensitizer in PDT in the 1980s. A mixture of chlorin e6 and hematoporphyrin derivatives was found to be present in cell membranes such as the cytoplasm and mitochondrial membranes. If the photosensitizer is specific to a surface antigen/receptor surface of a target cell and is bound to an antibody or ligand, a photosensitive action may be increased.

There have been the results of allowing chlorin e6 to act on cancer cells together with a monoclonal antibody, which may increase the selectivity of porphyrin photosensitive actions by reacting specifically on cancer cell surfaces. The target cell of this interaction may be a cell membrane. For example, the sensitivity to light exposure shows that intracellular organs such as the nucleus and lysosomes are likely to be much more sensitive targets. As will be described later, according to the present invention, a photodynamic therapy method mediated by a chlorin e6 photosensitizer composite for treating obesity may be effective for the treatment of obesity.

According to one embodiment of the present invention, a photodynamic therapy method including one or more of chlorin K, chlorin P6, rhodochlorin, perpurin 18, chlorin e4, rhodin G7, porphyrin K, and porphyrin e4 among chlorin e6 and/or compounds derived from chlorin e6 and using a composite formed with additives such as polyvinylpyrrolidone as a photosensitizer may be effective in treating obesity.

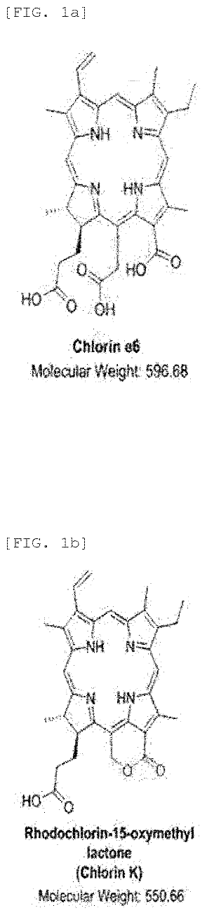

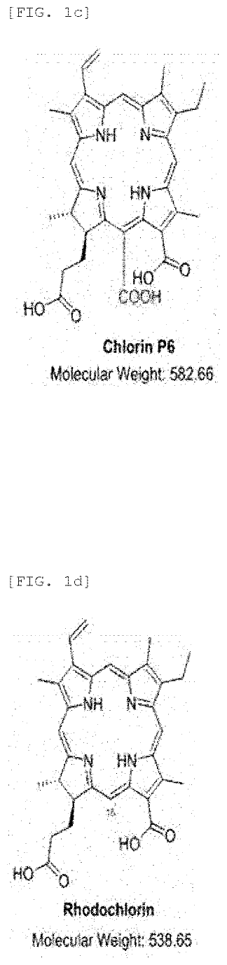

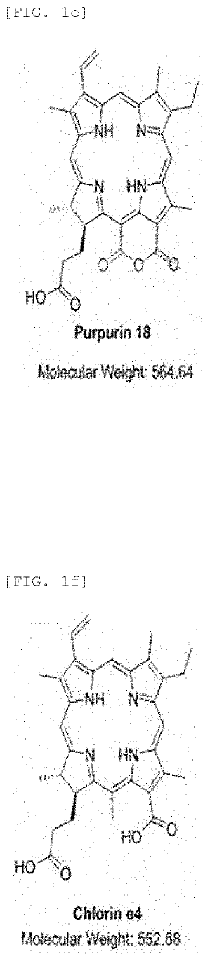

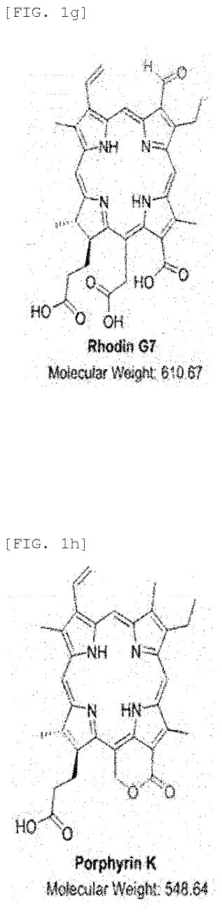

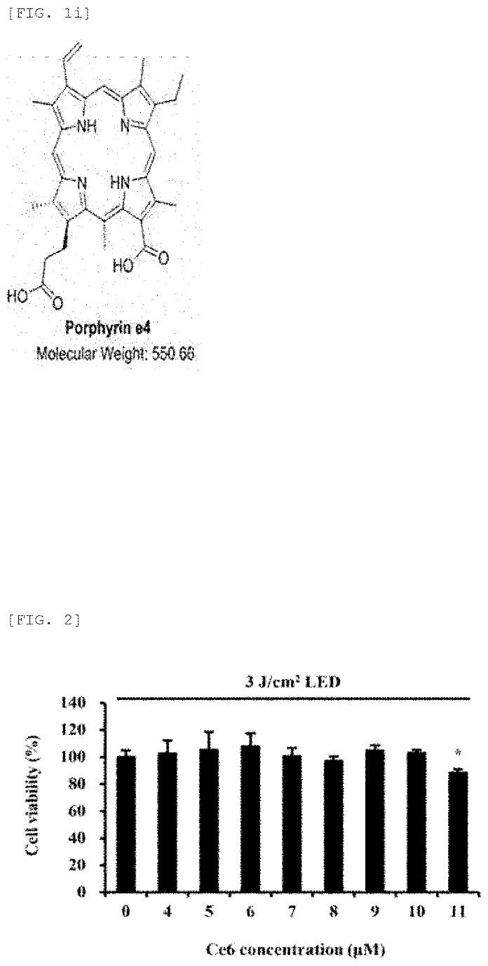

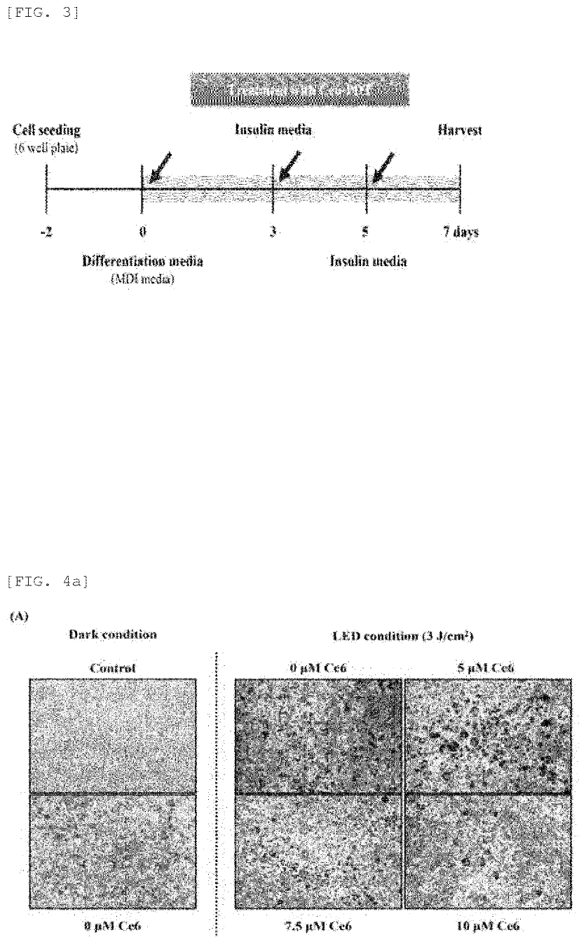

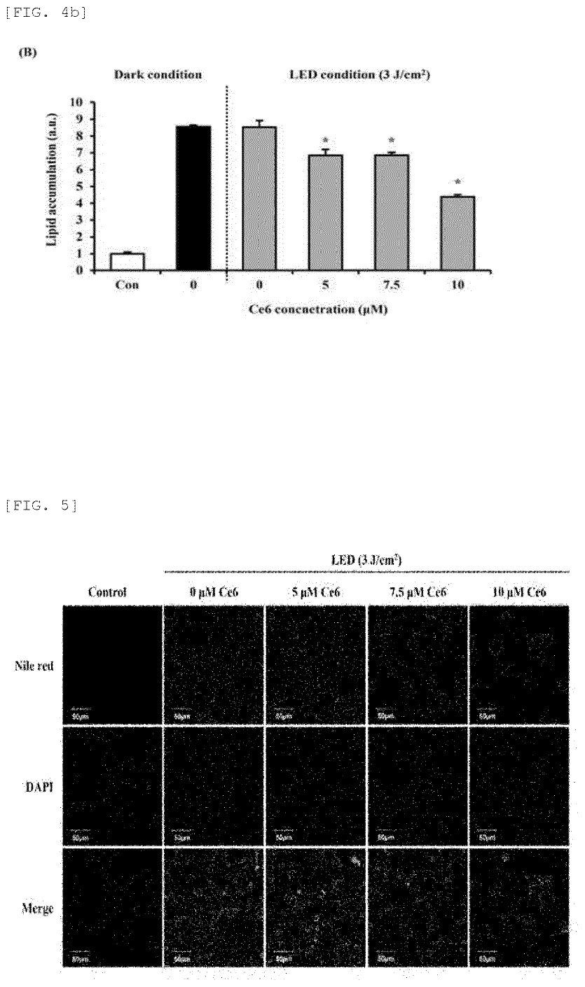

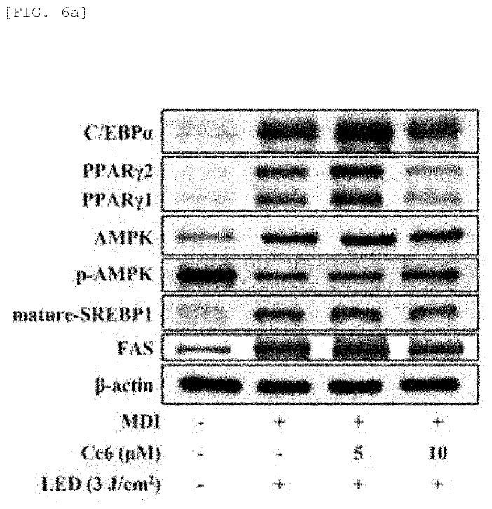

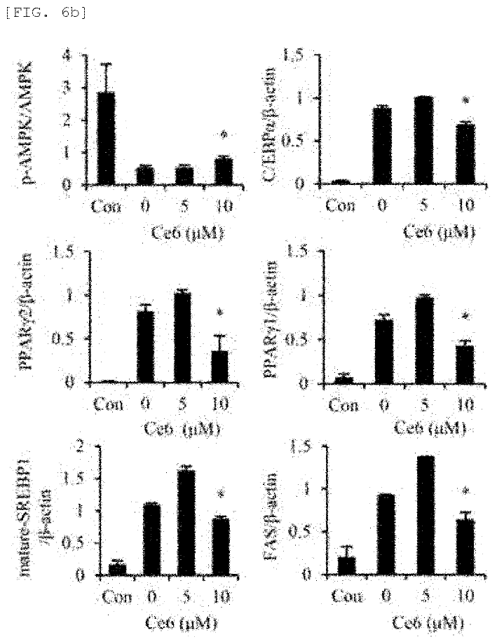

According to one embodiment of the present invention, the structures for chlorin K, chlorin P6, rhodochlorin, perpurin 18, chlorin e4, rhodin G7, porphyrin K, or porphyrin e4 are shown in FIGS. 1A to 1I, respectively

In the present specification, the term “chlorin e6 composite” may refer to a composite configured to include chlorin e6 and at least one or more chlorin e6 derivatives.

In one embodiment, the “chlorin e6 composite” may be configured to include chlorin e6 and at least one or more chlorin e6 derivatives. As one example, the “chlorin e6 composite” may be configured to include one or more of chlorin e6, chlorin K, chlorin P6, rhodochlorin, perpurin 18, chlorin e4, rhodin G7, porphyrin K, and porphyrin e4. For example, the chlorin e6 composite may be configured to include chlorin e6 and chlorin K. For example, the chlorin e6 composite may be configured to include chlorin e6 and porphyrin K. As another example, the chlorin e6 composite may be configured to include chlorin e6 and chlorin P6. As still another example, the chlorin e6 composite may be configured to include chlorin e6 and chlorin P6. In this way, the chlorin e6 composite may be configured to include chlorin e6, but further include at least one or more chlorin e6 derivatives.

In another embodiment, the “chlorin e6 composite” may be configured to include chlorin e6, at least one or more chlorin e6 derivatives, and additives. The additive may be, for example, polyvinylpyrrolidone, but is not limited thereto. The chlorin e6 composite may be configured to include, for example, chlorin e6, rhodin G7 and additives. For example, the chlorin e6 composite may be configured to include chlorin e6, porphyrin K and additives. As another example, the chlorin e6 composite may be configured to include chlorin e6, chlorin P6 and additives. As still another example, the chlorin e6 composite may be configured to include chlorin e6, chlorin P6, rhodochlorin and additives. In this way, the chlorin e6 composite may be configured to include chlorin e6, but further include at least one or more chlorin e6 derivatives and additives.

According to one embodiment of the present invention, a photodynamic therapy method mediated by a chlorin e6 photosensitizer composite for treating and preventing obesity may use the chlorin e6 composite described above. For example, a weight may be reduced by intravenously injecting or orally administering the chlorin e6 composite to a subject for photodynamic therapy. In addition, the chlorin e6 composite may be intravenously injected or orally administered to the subject for photodynamic therapy, thereby preventing or treating the subject's obesity and metabolic diseases. [43] According to embodiments of the present invention, the chlorin e6 composite may be administered to the subject for photodynamic therapy, so as to reduce triglycerides, decrease low-density proteins, and increase high-density proteins, thereby treating the subject's obesity.

According to embodiments of the present invention, the chlorin e6 composite may be intravenously injected or orally administered to the subject so as to perform a certain intensity of photodynamic therapy several times within four weeks or every week, thereby treating the subject's obesity.

According to embodiments of the present invention, the subject's obesity may be treated by administering the chlorin e6 composite in an amount of 1.0 mg to 10.0 mg per 1 kg of the subject and performing photodynamic therapy. For example, if the subject's weight is 50 kg, the chlorin e6 composite may be administered to the subject in an amount of 50.0 mg to 500.0 mg.

According to embodiments of the present invention, the subject's obesity may be treated by administering the chlorin e6 composite to the subject, exposing the subject to an LED laser having a wavelength of 660 nm and an intensity of 1.0 J/cm² to 5.0 J/cm² and performing photodynamic therapy.

According to embodiments of the present invention, the subject's obesity may be treated by administering the chlorin e6 composite configured to include chlorin e6 and at least one or more chlorin e6 derivatives (for example, at least one or more of chlorin K, chlorin P6, rhodochlorin, perpurin 18, chlorin e4, rhodin G7, porphyrin K, and porphyrin e4) to the subject and performing photodynamic therapy.

According to embodiments of the present invention, the subject's obesity may be treated by administering the chlorin e6 composite configured to include chlorin e6, at least one or more chlorin e6 derivatives (for example, at least one or more of chlorin K, chlorin P6, rhodochlorin, perpurin 18, chlorin e4, rhodin G7, porphyrin K, and porphyrin e4) and additives such as polyvinylpyrrolidone to the subject and performing photodynamic therapy.

According to one embodiment of the present invention, there may be provided a photodynamic therapy method mediated by a chlorin e6 photosensitizer composite for treating and preventing obesity, the method comprising: injecting or administering a chlorin e6 composite into a subject; and performing photodynamic therapy on the subject into which the chlorin e6 composite has been injected or administered.

According to another embodiment of the present invention, there may be provided a photodynamic therapy method mediated by a chlorin e6 photosensitizer composite for treating and preventing a subject’ metabolic diseases, the method comprising: injecting or administering a chlorin e6 composite into a subject; and performing photodynamic therapy on the subject into which the chlorin e6 composite has been injected or administered.

Hereinafter, the results of experimenting on a photodynamic therapy method mediated by a chlorin e6 photosensitizer composite for treating and preventing obesity according to one embodiment of the present invention will be described with reference to drawings.

<Materials and Methods>

1) Preparation of MDI Medium for Cell Culture and Differentiation

3T3-L1 adipocyte precursors were purchased from ATCC in the United States. Dulbecco's modified Eagle's medium (DMEM) containing 10% fetal bovine serum and 1% penicillin-streptomycin was added to the cells and cultured. Then, for differentiation, 3T3-L1 cells were added into a 6-well plate at 1×10⁵ cells and a medium containing 0.5 mM 3-isobutyl-1-methyl xanthine (IBMX), 1 μM dexamethasone, and 10 μg/mL insulin was used to induce differentiation. In three days later, the MDI medium was replaced with a medium containing 10 μg/mL insulin and 10% fetal bovine serum, and was replaced with a fresh medium once every two days until the cells were used for an experiment.

2) LED for PDT Experiment Mediated by Composite Composed of Chlorin e6 and/or Chlorin e6 Derivatives

Chlorin e6 composites at various concentrations were added into 3T3-L1 cultured on a 6-well plate and treated in the dark at 37° C. and 5 CO₂ for 30 minutes. After three hours in the dark, above 3T3-L1 containing chlorin e6 composites at various concentrations were exposed to an LED laser having a wavelength of 660 nm and an intensity of 3 J/cm². The medium containing the chlorin e6 composite was replaced with the MDI medium and incubated in the dark for three days. And on the 3rd day, the cells were treated with photodynamic therapy (hereinafter also referred to as “PDT”) mediated by the chlorin e6 composite in the same manner as on an initial day. Then, replacement was made with a medium containing 10 μg/mL of insulin and 10% fetal bovine serum, followed by incubating in the dark for two days and performing PDT mediated by the chlorin e6 composite once more on the 5th day. And, the cells were harvested on the 7th day.

3) MTT Experiment

3T3-L1 cells are incubated in a 96-well plate at 37° C. and 5% CO₂ in the dark. The cells were treated with 4 to 10 μM of the chlorin e6 composite for 30 minutes at 37° C. and 5% CO₂ in the dark. And in three hours later, the cells were exposed to an LED laser having a wavelength of 660 nm and an intensity of 3 J/cm². After that, the medium was replaced with a medium without serum added and incubated for 44 hours. Then, after formazan was formed, dimethyl sulfoxide (DMSO) was added and absorbance was measured at 570 nm.

4) Determination of Lipid Content Through Oil-Red O Staining

Oil-red O solution was prepared after being purchased from Sigma, USA, and isopropyl alcohol was diluted in water at a ratio of 3:2. Mature 3T3-L1 cells were fixed in 10% formalin for 10 minutes at room temperature. After formalin was washed, the 3T3-L1 cells were stained by being placed in oil-red O solution at room temperature for one hour and washed with 601 isopropyl alcohol. The images of the stained cells were obtained by using a CCD Pro 2 camera from IMT I-Solution Inc., in the United States. The images of cell morphology were obtained by using a microscope. Cells stained with lipid grains were eluted in isopropanol and were seeded at 200 μL into a 96-well plate. An absorbance of each well was measured at a wavelength of 420 nm by using Sunrise of TECAN, a Swiss ELISA analyzer.

5) Nile Red Staining

3T3-L1 cells were incubated in a culture dish of a 60 mm confocal light microscope. The 3T3-L1 cells were induced into differentiation to perform PDT mediated by chlorin e6, and the cells were fixed with 10% formalin at room temperature for 10 minutes. After that, formalin was washed clean, stained with nile red from Sigma USA by being placed therein for one hour at room temperature, and washed with PBS in the dark. And after staining with DAPI (4′,6-diamidino-2-phenylindole) for five minutes and washing with phosphate buffer solution (PBS), an observation was made by using an FV10-ASW confocal light optical microscope from Olympus in Japan.

6) Western Blot Analysis

The whole cell lysate was prepared by using an RIPA buffer containing phenylmethylsulfonyl fluoride (PMSF) and protease inhibitor. All of the samples were prepared by adding 25 μg of proteins to 5×SDS-PAGE loading buffer, followed by separating proteins through SDS-PAGE. Then, the proteins in the separated gel were transferred to a polyvinylidene fluoride (PVDF) membrane and placed in 5# bovine serum albumin (BSA) for two hours. The PVDF membrane blocked by using the BSA was allowed to bind each of primary antibodies at 4° C. for 18 hours, and left alone at room temperature for 50 minutes so that the secondary antibody to which horseradish peroxidase (HRP) is attached may bind to the primary antibody at room temperature for 50 minutes. This PVDF membrane was activated with Supernova ECL western detection solution and images were obtained by using Sensi-Q 2000 from Lugen sci Co., in Korea. The quantification of the images obtained was performed by using the imageJ program.

7) RT-PCR

Total RNA was isolated by harvesting 3T3-L1 cells, and cDNA was synthesized from 1 μg of total RNA by using a cDNA kit from Bio-rad, in the USA. Synthesis conditions were set at 25° C. for five minutes, at 46° C. for 20 minutes, and at 95° C. for one minute as one cycle. For polymerase chain reaction (PCR) amplification, DNA was amplified by setting 25 cycles of 95° C. for five minutes, 95° C. for 30 seconds, 60° C. for 30 seconds, 72° C. for one minute, and 72° C. for five minutes. The PCR products were subjected into electrophoresis in a 2% agarose gel. The primers used in this experiment are as follows.

C/FEBPα
Forward:
5′-TGTTGGGGATTTGAGTCTGTG-3′,
Reverse:
5′-GGAAACCTGGCCTGTTGTAAG-3′
FAS
Forward:
5′-GCTGCGGAAACTTCAGGAAAT-3′,
Reverse:
5′-AGAGACGTGTCACTCCTGGACTT-3′
LPL
Forward:
5′-GGGAGTTTGGCTCCAGAGTTT-3′,
Reverse:
5′-TGTGTCTTCAGGGGTCCTTAG-3′.

8) Experiment on Anti-Obesity Effect of PDT Mediated by Chlorin e6 Composite Using Mice with Obesity Induced

Six-week-old female ICR mice were purchased from Orient Bio in Korea, acclimated for one week, and fed with a high-fat diet containing 60% fat until the end of the experiment. The experiment started on the 15th day when the weight of each individual was increased by about 20% compared to the initial weight. The experimental group consists of a total of seven groups as follows.

Group 1—normal food diet (NFD)

Group 2—high fat diet (HFD)

Group 3—HFD+chlorin e6 composite (2.5 mg/kg)

Group 4—HFD+low LED (3 J/cm²)

Group 5—HFD+high LED (5 J/cm²)

Group 6—HFD+chlorin e6 composite (2.5 mg/kg)+low LED (3 J/cm²)

Group 7—HFD+chlorin e6 composite (2.5 mg/kg)+high LED (5 J/cm²)

The chlorin e6 composite was applied through an intraperitoneal injection and the LED was irradiated for three hours after injection. The above experiment was applied at two-day intervals.

9) Statistical Processing

The data were subjected into the statistical processing program (statistical package for social science) of SPSS Inc., in the USA. Each of three independent experimental results that can be representative was subjected into the program so as to obtain an average value and a standard deviation of each group. The statistical significance was calculated through one-way ANOVA and was obtained by Tukey's post hoc method. It can be considered as statistically significant when P<0.05.

10) CT Scan for Measuring Fat Mass of Beagle Dogs

Twelve female beagle dogs which were eleven months old were purchased from Orient Bio Jeongeup Center in Korea and put into room 1 of a first animal breeding area which was set to a temperature of 23±3° C., relative humidity of 55±15%, ventilation frequency of 10-20 times/hr, lighting time of 12 hours (turned on at 8 a.m. and turned off at 8 p.m.), and illuminance of 150-300 Lux. For feed, each of the dogs was allowed to take about 300 g of feed for experimental animals which were produced by Cargill Agripurina and supplied from Biopia in Korea, and was also allowed to have a free access to water through an automatic watering device. The test group was divided into a total of four groups.

Group 1—high fat diet (HFD)

Group 2—HFD+dosed with chlorin e6 composite

Group 3—HFD+dosed with chlorin e6 composite+irradiated with LED

Group 4—HFD+dosed with chlorin e6 composite+irradiated with LED

2.5 mg/kg of chlorin e6 composite was intravenously injected by using an infusion machine for 30 minutes, and the LED laser was irradiated with an intensity of 7.34 mW/cm².

The chlorin e6 composite was administered three times a week until the 16th day, in which LED laser irradiation was performed for 15 minutes (group 3; 6.6 J/cm²) and 30 minutes (group 4; 13.2 J/cm²). After that, the chlorin e6 composite was administered five times a week, and the LED laser was irradiated for 10 minutes (group 3; 4.4 J/cm²) and 15 minutes (group 4; 6.6 J/cm²). CT image was scanned by using Alexion of Toshiba Corporation in Japan before administration of the test substance (Day 0) and on the day of autopsy (day 35) after anesthesia using 5 mg/kg of Zoletil 50 of VIRBAC in France and 2.5 mg/kg of Rompun of Bayer AG in Germany.

<Results>

1) Efficacy of Viability of 3T3-L1 Cells Using Composite Composed of Chlorin e6 or Chlorin e6 Derivatives

3T3-L1 cells were treated with chlorin e6 composite diluted at a concentration of 4 to 11 μM, and MTT analysis was performed. The cells showed a viability of about 88? in 48 hours after being treated with PDT mediated by 11 μM of the chlorin e6 composite. Cytotoxicity was not significant in the group treated with the chlorin e6 composite at a concentration of 11 μM or less.

2) Relationship Between Lipid Synthesis in 3T3-L1 Cell Differentiation Induced by MDI Medium and PDT Mediated by a Composite Composed of Chlorin e6 or Chlorin e6 Derivatives

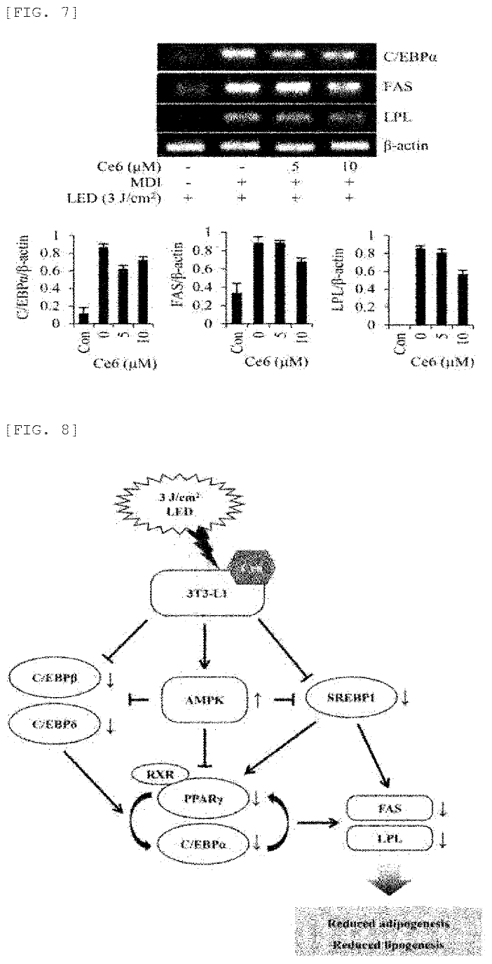

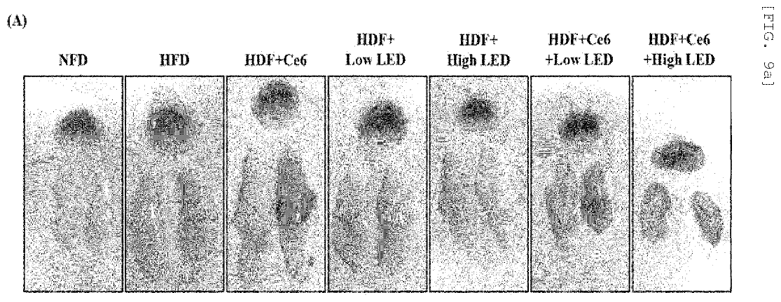

PDT mediated by the chlorin e6 composite was performed three times by the same procedure as in FIG. 3. And, with regard to the differentiated 3T3-L1 cells, as a result of confirming through oil-red 0 staining if PDT treatment mediated by the chlorin e6 composite has an effect on adipocyte differentiation, small, long and fat granules were not observed in the control group before differentiation was induced, but round and fat granules were observed when differentiation was induced through MDI medium. However, with regard to the group treated with the PDT mediated by the chlorin e6 composite, it could be confirmed that the induction of differentiation through the MDI medium is suppressed as a concentration increases. In addition, it could be confirmed that 3T3-L1 cells are differentiated similarly to those of the control group if the LED is not irradiated with or without the chlorin e6 composite. With regard to the group treated with the PDT mediated by the chlorin e6 composite at a concentration of 5 to 7.5 μM, it could be confirmed that fat is reduced by about 20%, whereas the accumulation of fat is suppressed by about 60% at a concentration of 10 μM.

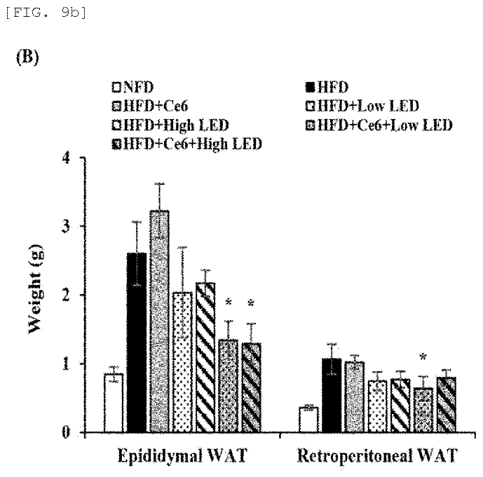

In addition, referring to FIG. 5, as a result of staining a lipid overload of 3T3-L1 with nile red, it could be confirmed that the synthesis of triglycerides decreases according to the concentration of the chlorin e6 composite in the group treated with the PDT mediated by the chlorin e6 composite.

3) Relationship Between mRNA Expression of Proteins Involved in Production of Fats and Lipids and PDT Mediated by Composite Composed of Chlorin e6 or Chlorin e6 Derivatives

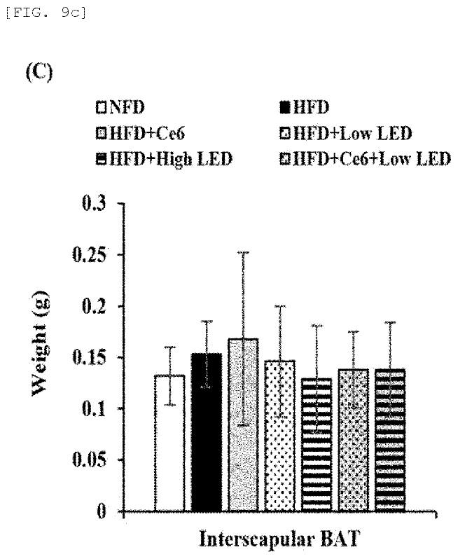

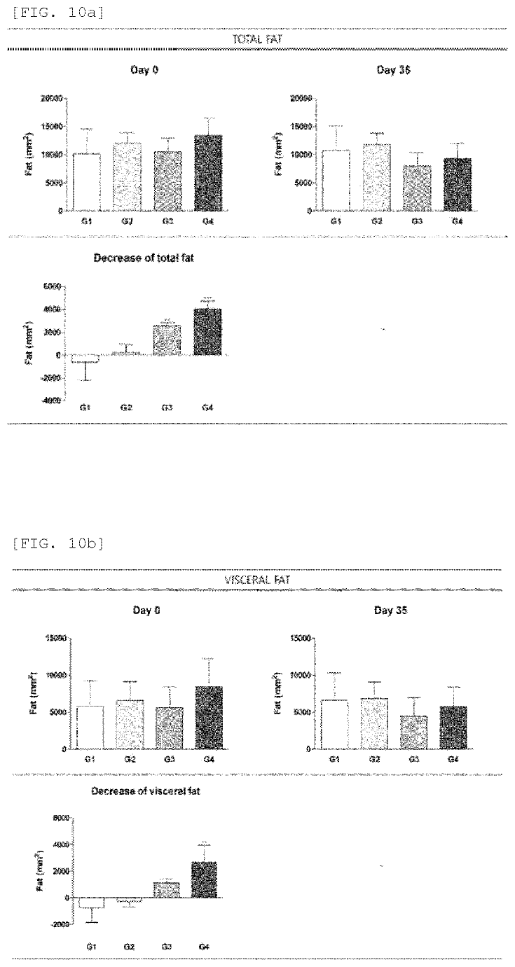

To confirm the effect of the PDT mediated by the chlorin e6 composite from a molecular biological point of view, the expression levels of PPARγ and C/EBPa, which are factors related to adipogenesis, were confirmed by Western blot using differentiated 3T3-L1 cells. Referring to FIG. 6, it can be understood that the expression levels of PPARγ-1 and 2 and C/EBPa decrease when the LEDs were irradiated after treatment with 10 μM of the chlorin e6 composite. It could be also confirmed that the expression level of SREBP-1, which regulates fat absorption and biosynthesis, decreases in the group treated with the PDT mediated by the chlorin e6 composite. It could be confirmed that phosphorylated AMPK is remarkably increased.

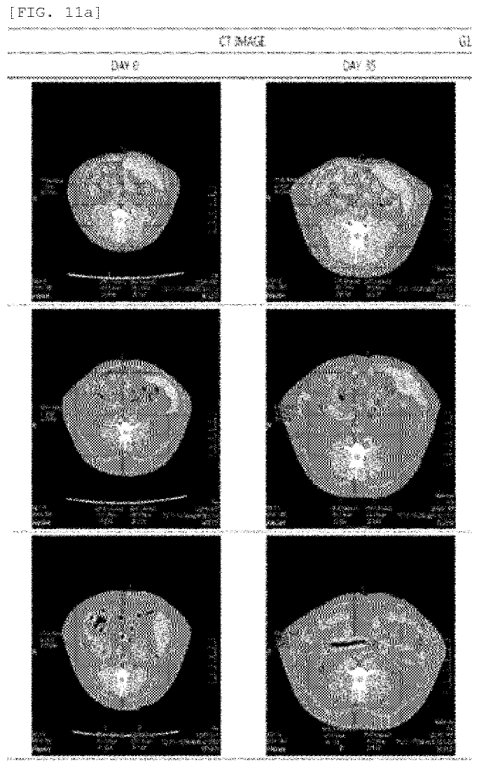

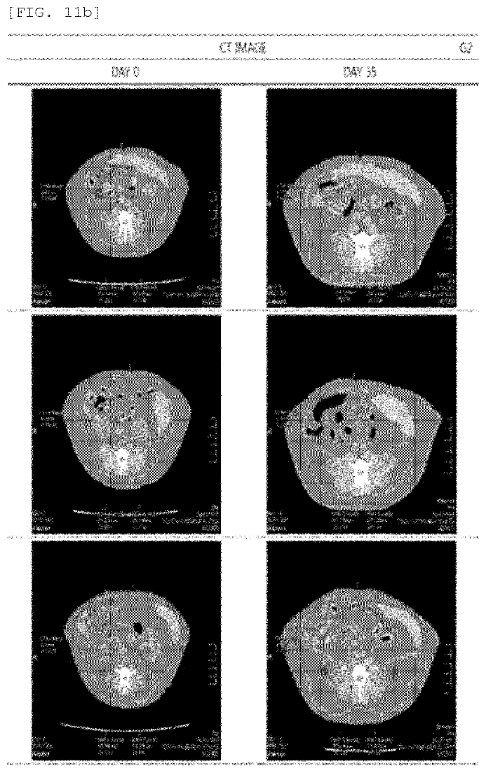

RT-PCR was performed to confirm if the PDT mediated by the chlorin e6 composite also affects the expression level of mRNA involved in fat and lipogenesis. Referring to FIG. 7, it could be confirmed that the expression level of LPL mRNA is remarkably decreased at a concentration of 10 μM of the chlorin e6 composite. In addition, it could be confirmed that the mRNA expression levels of C/EBPa and FAS are also decreased. However, the PDT mediated by the chlorin e6 composite has no effect on undifferentiated 3T3-L1 cells.

4) Relationship Between Weight Change of WAT and BAT in Mice with Obesity Induced and PDT Mediated by Composite Composed of Chlorin e6 or Chlorin e6 Derivatives

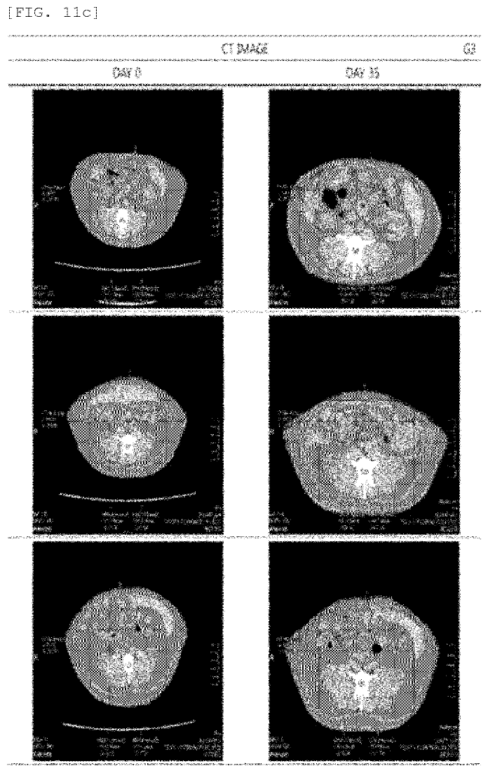

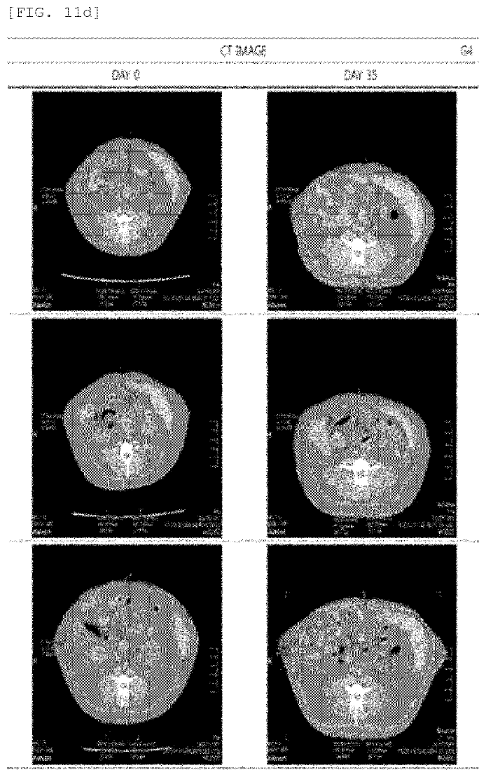

To observe a weight change in the white fat and brown fat of obese mice, the WAT of epididymis and the BAT of scapula were extracted to be compared in size, and a weight was measured. When observing the extracted adipose tissue with the naked eye as shown in FIG. 9A, it was confirmed that the white fat of the group fed with a high fat diet (HFD) is enlarged compared to the group fed with a normal food diet (NFD) and a weight difference is also quite large. Normal Food Diet) [symbol] (HFD: High Fat Diet)

[symbol]

[symbol]

[symbol]

It could be confirmed that the white fat of the group treated with the chlorin e6 composite and irradiated with low LED and high LED is remarkably smaller compared to the group not treated therewith and a weight is also considerably decreased (by about 50%). Meanwhile, it could be confirmed that there is little or no change in the size or weight of white fat in the group treated with the chlorin e6 composite only. However, in the case of the scapular BAT, no significant change was observed in all the groups, and thus it is considered that the PDT mediated by the chlorin e6 composite does not have a significant effect on the weight of brown fat.

5) Anti-Obesity Effect of PDT Mediated by Composite Composed of Chlorin e6 or Chlorin e6 Derivatives on Beagle Dogs with Obesity Induced

Beagle dogs were used to carry out an experiment on the fat reduction effect of the PDT mediated by the chlorin e6 composite from an in vivo perspective. A CT scan was performed before the start of the experiment, and a CT scan was performed again on the last day of the experiment to measure the fat mass. As a result, it was confirmed that a total fat reduction and a visceral fat reduction in group 4 are significantly higher than those of the untreated group. Thus, although the PDT mediated by the chlorin e6 composite appears to have an obesity treatment effect on beagle dogs, it could be confirmed that the effect of alleviating obesity is insignificant when the LED laser is not irradiated after treatment with the chlorin e6 composite alone, and it could be also confirmed that the effect of alleviating obesity increases as the time of irradiating the LED laser increases.

### REFERENCE

1. Kim Y M, Kim I H, Choi J W, Lee M K, Nam T J. The anti-obesity effects of a tuna peptide on 3T3-L1 adipocytes are mediated by the inhibition of the expression of lipogenic and adipogenic genes and by the activation of the Wnt/beta-catenin signaling pathway. Int J Mol Med. 2015; 36(2):327-34. Epub 2015/06/06. doi: 10.3892/ijmm.2015.2231. PubMed PMID: 26046125; PubMed Central PMCID: PMCPMC4501660.
2. Ondrusova K, Fatehi M, Barr A, Czarnecka Z, Long W, Suzuki K, et al. Subcutaneous white adipocytes express a light sensitive signaling pathway mediated via a melanopsin/TRPC channel axis. Sci Rep. 2017; 7(1):16332. Epub 2017/11/29. doi: 10.1038/s41598-017-16689-4. PubMed PMID: 29180820; PubMed Central PMCID: PMCPMC5703708.
3. Takahata T, Kumano T, Ookawa K, Hayakari M, Kakizaki I, Tsuchida S. Inhibition of 3T3-L1 adipocyte differentiation by 6-ethoxyzolamide: repressed peroxisome proliferator-activated receptor gamma mRNA and 345 enhanced CCAAT/enhancer binding protein beta mRNA levels. Biochem Pharmacol. 2004; 67(9):1667-75. Epub 2004/04/15. doi: 10.1016/j.bcp.2003.12.039. PubMed PMID: 15081866.
4. Lai J, Liu G, Yan G, He D, Zhou Y, Chen S. WEHI-3 cells inhibit adipocyte differentiation in 3T3-L1 cells. Biochem Biophys Res Commun. 2015; 462(2):105-11. Epub 2015/04/26. doi: 10.1016/j.bbrc.2015.04.064. PubMed PMID: 25911323.
5. Madsen L, Petersen R K, Sorensen M B, Jorgensen C, Hallenborg P, Pridal L, et al. Adipocyte differentiation of 3T3-L1 preadipocytes is dependent on lipoxygenase activity during the initial stages of the differentiation process. Biochem J. 2003; 375(Pt 3):539-49. Epub 2008/03/07. PubMed PMID: 18320708.
6. Cho E J, Rahman M A, Kim S W, Baek Y M, Hwang H J, Oh J Y, et al. Chitosan oligosaccharides inhibit adipogenesis in 3T3-L1 adipocytes. J Microbiol Biotechnol. 2008; 18(1):80-7. Epub 2008/02/02. PubMed PMID: 18239421.
7. Kim S J, Nian C, McIntosh C H. Activation of lipoprotein lipase by glucose-dependent insulinotropic polypeptide in adipocytes. A role for a protein kinase B, LKB1, and AMP activated protein kinase cascade. J Biol Chem. 2007; 282(12):8557-67. Epub 2007/01/25. doi: 10.1074/jbc.M609088200. PubMed PMID: 17244606.
8. Kang J W, Nam D, Kim K H, Huh J E, Lee J D. Effect of Gambisan on the inhibition of adipogenesis in 3T3-L1 adipocytes. Evid Based Complement Alternat Med. 2013; 2013:789067. Epub 2013/09/27. doi: 10.1155/2013/789067. PubMed PMID: 24069055; PubMed Central PMCID: PMCPMC3773429.
9. Zhou W, Han W F, Landree L E, Thupari J N, Pinn M L, Bililign T, et al. Fatty acid synthase inhibition activates AMP-activated protein kinase in SKOV3 human ovarian cancer cells. Cancer Res. 2007; 67(7):2964-71. Epub 2007/04/06. doi: 10.1158/0008-5472.CAN-06-3439. PubMed PMID: 17409402.
10. Statistics Canada. Canadian Community Health Survey 2004

1. A photodynamic therapy method mediated by a chlorin e6 photosensitizer composite for treating and preventing obesity, the method comprising:
injecting or administering a chlorin e6 composite into a subject; and
performing photodynamic therapy on the subject into which the chlorin e6 composite has been injected or administered.

2. The photodynamic therapy method of claim 1, wherein the injecting or administering of the chlorin e6 composite to the subject; and the performing of photodynamic therapy on the subject into which the chlorin e6 composite has been injected or administered are periodically carried out twice or more.

3. The photodynamic therapy method of claim 1, wherein the injecting or administering of the chlorin e6 composite to the subject is administering the chlorin e6 composite in an amount of 1.0 mg to 10.0 mg per 1 kg of the subject.

4. The photodynamic therapy method of claim 1, wherein the performing of photodynamic therapy is to expose the subject to an LED laser having a wavelength of 660 nm and an intensity of 1.0 J/cm² to 5.0 J/cm².

5. The photodynamic therapy method of claim 1, wherein the chlorin e6 composite comprises at least one of chlorin K, chlorin P6, rhodochlorin, perpurin 18, chlorin e4, rhodin G7, porphyrin K, and porphyrin e4 among chlorin e6 and compounds derived from chlorin e6.

6. A photodynamic therapy method mediated by a chlorin e6 photosensitizer composite for treating and preventing obesity, wherein the chlorin e6 composite comprises at least one of chlorin K, chlorin P6, rhodochlorin, perpurin 18, chlorin e4, rhodin G7, porphyrin K, and porphyrin e4 among chlorin e6 and compounds derived from chlorin e6, and additives.

7. A photodynamic therapy method mediated by a chlorin e6 photosensitizer composite for treating and preventing a subject's metabolic diseases, the method comprising:
injecting or administering a chlorin e6 composite into the subject; and
performing photodynamic therapy on the subject into which the chlorin e6 composite has been injected or administered.
